(19) United States
(12) Patent Application Publication
(10) Pub. No.: US 2009/0099145 A1
(43) Pub. Date: Apr. 16, 2009

(54) Process for Producing a Single-phase Pharmaceutical Preparation for Limiting/Reducing the Risk of Deep Venous Thrombosis in Association with Oral Contraception

(76) Inventor: Katrin Mittmann, Jena (DE)

Correspondence Address: STRIKER, STRIKER & STENBY, 103 EAST NECK ROAD, HUNTINGTON, NY, 11743, US

(21) Appl. No.: 12/249,167

(22) Filed: Oct. 10, 2008

Related U.S. Application Data
(60) Provisional application No. 60/979,210, filed on Oct. 11, 2007.

(51) Int. Cl. A61K 31/565; A61P 7/02

(52) U.S. Cl. 514/170

(57) ABSTRACT

Single-phase oral contraceptives containing a combination of 2.0 mg of dienogest and 0.030 mg of ethinylestradiol or 2.0 mg of dienogest and 0.020 mg of ethinylestradiol in n×21 daily dose units followed by n×21 of at the most 7 daily hormone-free or placebo-containing dose units, where n equals 2, 3, 4, 5, 6, 7, 8, 9, 10, 11, 12, 13, 14, 15, 16 and 17, bring about a limitation/reduction of the risk of deep venous thrombosis in combination with oral contraception.

A suitable contraceptive agent with a prolonged intake period of the hormone-containing daily dose units is provided which despite the prolonged intake period keeps the risk of a deep venous thrombosis at the same level as with a conventional oral contraceptive and thus within reasonable limits.

### TECHNICAL FIELD

The invention relates to a process for producing a single-phase pharmaceutical preparation for limiting/reducing the risk of deep venous thrombosis in association with oral contraception. A contraceptive combination containing 2.0 mg of 17-α-cyanomethyl-17-β-hydroxyestra-4,9-dien-3-one (dienogest) and 0.030 mg of 17-α-ethinylestradiol (ethinylestradiol) or 2.0 mg of dienogest and 0.020 mg of ethinylestradiol is used in n×21 daily dose units, the n×21 units being followed by at the most 7 hormone-free or placebo-containing daily dose units, with n equal to 2, 3, 4, 5, 6, 7, 8, 9, 10, 11, 12, 13, 14, 15, 16 and 17.

### PRIOR ART

It is known that oral contraceptives can increase the risk of venous thrombolytic disorders three to four times (Birkhäuser. M., et al.: Recommendations for Hormonal Contraception—34th Meeting of the “Zurich Discussion Group”, April 2005, Gyn. Geburtshilfl. Rundsch. 2006; 46:58-63).

Venous thromboembolisms occur rarely in young women. For women below the age of 45 years who do not use contraceptives, 5 to 10 such events are estimated to occur per 100,000 woman-years. The ingestion of contraceptives increases the risk (Birkhäuser. M., et al.: Recommendations for Hormonal Contraception—34th Meeting of the “Zurich Discussion Group”, April 2005, Gyn. Geburtshilfl. Rundsch. 2006; 46:58-63). It has also been found that the risk depends on the dose of ethinylestradiol, and diverse observation studies have shown that oral contraceptives containing progestogens of the so-called third generation, such as desogestrel and gestoden, could be associated with a higher risk than oral contraceptives containing progestogens of the so-called second generations, such as levonorgestrel (Weiss, G., Risk of venous thromboembolism with third-generation oral contraceptives: A review, Am. J. Obstet. Gynecol. 1999: 180: 295-301).

The relative risk of deep venous thrombosis (DVT) increases particularly during the first months of use of conventional oral contraceptives (21 days of using hormonal daily dose units) indicating that predisposition and risk factors play an important role. Because, however, DVT occurs in young women only rarely, sufficiently reliable epidemiological events cannot be expected before new preparations have been used by a large number of women for a sufficiently long time. As compensation, the permit-issuing authorities require controlled studies of the action of new oral contraceptives or ingestion regimens on the hemostasis parameters. Most hemostasis factors are formed in the liver and could be modified by oral by oral contraceptives. They are affected mostly by ethinylestradiol, whereas progestogens could partly weaken the estrogenic action. For this reason, the effects on many hemostasis variables depend on the ethinylestradiol dose, and the various progestogens could differ in terms of their modifying action on the estrogen-dependent hemostasis parameters. The progestogens are not a homogeneous class of hormones; rather, they differ in their hormonal activity pattern. As for their effect on the coagulation and fibrinolysis, progestogens with androgenic properties such as levonorgestrel could inhibit the action of ethinylestradiol on some hemostasis factors, whereas progestogens without androgenic activity, for example chlormadinone acetate or cyproterone acetate, are without effect. Regardless of their effect on the hemostasis, ethinylestradiol and the various progestogens, however, could directly affect the function of the endothelium and of the smooth muscle cells. An example of this are certain progestogens such as medroxyprogesterone acetate, gestoden or 3-ketodesogestrel, which can regulate to a high degree the thrombin receptor and tissue factor and, hence, the procoagulatory activity in the vessel wall.

Although oral contraceptives can influence most hemostasis factors, a plausible biological explanation of the increased risk of thromboembolic disorders is missing, because some changes are prothrombolytic and others, on the other hand, may be viewed as antithrombotic. At any rate, there are some findings that could establish a connection between the particularities of hemostasis and the increased risk of thrombosis. The increase in factor VII and factor VIII and the reduction in antithrombin and protein S which are observed during the intake of oral contraceptives was more pronounced in women in whom DVT occurred during the use of oral contraceptives. The tissue factor-induced thrombin formation is markedly increased by the intake of oral contraceptives in women with a history of thrombosis, which points to prothrombotic anomalies. It is stated in the literature that the activation of procoagulatory factors by oral contraceptives could compensate for an increased fibrinolytic activity. On the other hand, the increased fibrinolytic potential could be counteracted by a more pronounced resistance to thrombolysis caused by a thrombin-activatable fibrinolysis inhibitor (TAFI) which resistance is increased by oral contraceptives, particularly by those containing third-generation progestogens.

The question remains whether a causal relationship exists between the lower DVT risk in women using oral contraceptives containing levonorgestrel rather than oral contraceptives containing gestoden, desogestrel or cyproterone acetate and the more pronounced estrogen-induced changes of the hemostasis parameters. It is being discussed whether the “estrogenicity” of the preparations, measured by the increased hepatic production of sexual hormone-binding globulin (SHBG), could be a measure of the risk of venous thromboembolic disorders.

The conventional ingestion regimen for oral contraceptives, which as a rule consists of 21 days of use of an estrogen/progestogen combination followed by a hormone-free interval of 7 days, was introduced about 45 years ago and is still the standard of hormonal pregnancy prevention. At any rate, there exist no medical reasons for regular withdrawal bleeding associated with the rapid decrease in serum concentration of contraceptive steroids during the pill-free week. This ingestion regimen was selected to imitate the natural cycle and to promote the general acceptance of this new contraception method.

On the other hand, regular cyclic fluctuations of the serum levels of contraceptively acting steroids, namely an increase during the first days of intake until a steady state is reached followed by a fast drop to the initial values during the hormone-free week, are not only associated with changes in many metabolic parameters, particularly the liver proteins, but also with possible somatic and psychic problems.

For this reason, women with cycle-dependent symptoms or menstruation-related symptoms have been practicing the omission of the hormone-free interval and the continuous use of oral contraceptives during several weeks or months and in some cases years. Various international surveys have, in fact, shown that most women prefer the prolonged intake to the conventional one.

To replace this off-label use of oral contraceptives with a treatment with approved intake regimens, long-cycle preparations have been developed which are supposed to reduce the frequent menstrual bleeding during the conventional use of oral contraceptives.

Many advantages of long-cycle intake of oral contraceptives are obvious or probable, but no sufficient data are as yet available concerning the risks associated with them.

### PRESENTATION OF THE INVENTION

The object of the invention is to provide suitable contraceptive means with an extended intake period of the hormone-containing daily dose units so that despite the extended intake period the risk of deep venous thrombosis associated with oral contraceptives is kept within justifiable limits.

According to the invention, this objective is reached by use of a process for producing a single-phase pharmaceutical preparation for limiting/reducing the risk of deep venous thrombosis in association with oral contraception which is a contraceptive combination containing 2.0 mg of 17-α-cyanomethyl-17-β-hydroxyestra-4,9-dien-3-one (dienogest) and 0.030 mg of 17-α-ethinylestradiol (ethynilestradiol) or 2.0 mg of dienogest and 0.020 mg of ethinylestradiol used in n×21 daily dose units the n×21 units being followed by at the most 7 hormone-free or placebo-containing daily dose units, with n equal to 2, 3, 4, 5, 6, 7, 8, 9, 10, 11, 12, 13, 14, 15, 16 and 17.

Advantageous embodiments of the invention are represented by the features described in claims 2 and 3.

The oral drug form can be a tablet, a tablet with a film coating (film-coated tablet) or a tablet with a sugar-containing covering.

Also included among the oral drug forms according to the invention are: hard gelatin capsules, soft gelatin capsules with an oily or aqueous suspension as filling material or with some other oral suspension. The release of the active ingredients, namely the dissolution of the said ingredients out of the tablet matrix/tablet core is determined by the dissolution test using 37° C. water as the dissolution medium at a rotation rate of 50 r.p.m.

The test is carried out in accordance with the European Pharmacopeia using a paddle agitator and 1000 mL of water.

Moreover, the objective is also reached by means of a kit according to claim 4.

The kit can also additionally contain 7, 6, 5, 4 or 3 hormone-free or placebo-containing daily dose units. These are intended for intake after a period of at least n×21 consecutive days, where n is equal to 2, 3, 4, 5, 6, 7, 8, 9, 10, 11, 12, 13, 14, 15, 16 and 17. Depending on the desire of the woman for continuous regulation of blood pressure and continuous contraception associated with the need for freedom from bleeding over a long period of time, the number of daily dose units containing the combination of dienogest and ethinylestradiol can amount to 84, with 7 hormone-free or placebo-containing daily dose units, so that the total number of cycle days per year is 4×(n×21+7), with n equal to 4.

It is known that the increase in hemostasis factors such as factor VII and factor VIII and the reduction in antithrombin and protein S, which are observed during the intake of oral contraceptives, is more pronounced in women in whom DVT appeared during the use of conventional oral contraceptives.

The study described in the following compares the time-dependent changes of various hemostasis plasma parameters during conventional treatment with ethinylestradiol/dienogest with the changes observed in women under long-cycle ethinylestradiol/dienogest treatment.

It is to be expected by those skilled in the art that the changes during uninterrupted treatment can be more pronounced than during conventional intake of oral contraceptives. In the course of a conventional oral contraceptive cycle, the hepatic production or estrogen-sensitive hemostasis factors during the first few days either increases (for example, fibrinogen, factor VII) or decreases (for example antithrombin) until a steady state is reached which recedes during the subsequent hormone-free interval. This pattern is repeated during all subsequent treatment cycles.

Surprisingly, the results of the present study show unequivocally that at no time during the treatment with ethinylestradiol/dienogest a significant difference between the conventional and the long-cycle use exists in terms of the hemostasis parameters.

These results indicate that the increase in procoagulatory and fibrinolytic activity observed after 3 months of use does not change substantially during the subsequent treatment. Moreover, the results show that the steady state of the estrogen-dependent changes is reached already after 3 months.

Hence, based on the results of the present study, we may conclude that to the extent that the EE/DNG-induced changes in hemostasis can be viewed as markers of the risk of venous thromboembolic disorders there is no reason to assume that long-cycle treatment presents a higher relative risk than the conventional use of EE/DNG.

### PRACTICAL EXAMPLES OF THE CONTRACEPTIVE COMBINATION

### Example 1

Valette is a conventional sugar-coated tablet for oral contraception containing 0.030 mg of ethinylestradiol and 2.0 mg of dienogest in a tablet core covered with a sugar-containing coating.

### Example 2

2 mg of dienogest and 0.02 mg of ethinylestradiol in which 1 mg of dienogest is released in delayed manner and 1 mg of dienogest and 0.02 mg of ethinylestradiol are released rapidly.

The example describes a film-coated tablet with a matrix core. The core of the film tablet contains 1 mg of dienogest in a hydrophilic erosion matrix with metolose as the base constituent. The matrix releases dienogest, the active ingredient, in retarded manner. The core was coated with a rapidly dissolving film containing 1.0 mg of dienogest and 0.02 mg of ethinylestradiol. For protection from light, the film tablet was covered with an additional rapidly dissolving iron oxide pigments-containing colored layer.

Composition
Core
Granulate 1

| | |
|---|---|
| Dienogest | 1.000 mg |
| Metolose 90 SH-4000 | 7.500 mg |
| Lactose monohydrate | 21.000 mg |
| Corn starch | 14.000 mg |
| Povidon K25 (10% in ethanol | 1.500 mg |

Granulate 2

| | |
|---|---|
| Lactose monohydrate | 54.000 mg |
| Corn starch | 27.100 mg |
| Maltodextrin (25% in water) | 6.900 mg |

Outer Phase

| | |
|---|---|
| Sodium carboxymethylstarch | 1.500 mg |
| Magnesium stearate | 1.500 mg |

Film Covering
Film 1 - containing active ingredient

| | |
|---|---|
| Dienogest | 1.000 mg |
| Ethinylestradiol | 0.020 mg |
| Methocel 5 | 2.250 mg |
| Talc | 0.450 mg |
| Titanium dioxide | 0.280 mg |

Film 2 - Colored Layer

| | |
|---|---|
| Methocel 5 | 3.375 mg |
| Talc | 0.675 mg |
| Titanium dioxide | 1.875 mg |
| Iron oxide, red | 0.075 mg |

### Example 3

Tablets having the following composition were prepared:

Core

| | |
|---|---|
| Dienogest | 2.000 mg |
| Ethinylestradiol | 0.030 mg |
| Metafolin | 0.451 mg |
| Lactose monohydrate | 28.720 mg |
| Corn starch | 15.000 mg |
| Maltodextrin | 3.750 mg |
| Magnesium stearate | 0.500 mg |

In place of ethinylestradiol, it is possible to use an ethinylestradiol-beta-cyclodextrin complex. The ethinylestradiol-beta-cyclodextrin complex (1:2) is used in an amount that is at the most, or about, ten times higher.

All substances were mixed in a suitable manner and granulated. Granulation was followed by metafolin application, repeated mixing, tableting and optionally film-coating.

Studies of the Efficacy of the Contraceptive Combination.

### DESCRIPTION OF THE FIGURES

The invention will now be described in greater detail by reference to the attached illustrations.

FIG. 1 shows the concentration of the procoagulatory parameter fibrinogen as a function of the time of use of the oral contraceptives, the control cycle indicating the concentration of fibrinogen when no oral contraceptive is used. In the figure, the striped segment represents the uninterrupted use during the long cycle (84 EE/DNG hormone-ingesting days+7 hormone-free days) and the full segment represents the conventional use (21 EE/DNG hormone-ingesting days+7 hormone-free days).

FIG. 2 shows the concentration of the procoagulatory parameter factor VII antigen as a function of the time of use of the oral contraceptives, the control cycle indicating the concentration of factor VII antigen when no oral contraceptive is used. In the figure, the striped segment represents the uninterrupted use during the long cycle (84 EE/DNG hormone-ingesting days+7 hormone-free days) and the full segment represents the conventional use (21 EE/DNG hormone-ingesting days+7 hormone-free days).

FIG. 3 shows the concentration of the procoagulatory parameter factor VII activity as a function of the time of use of the oral contraceptives, the control cycle indicating the concentration of factor VII activity when no oral contraceptive is used. In the figure, the striped segment represents the uninterrupted use during the long cycle (84 EE/DNG hormone-ingesting days+7 hormone-free days) and the full segment represents the conventional use (21 EE/DNG hormone-ingesting days+7 hormone-free days).

FIG. 4 shows the concentration of the procoagulatory parameter factor VIII activity as a function of the time of use of the oral contraceptives, the control cycle indicating the concentration of factor VIII activity when no oral contraceptive is used. In the figure, the striped segment represents the uninterrupted use during the long cycle (84 EE/DNG hormone-ingesting days+7 hormone-free days) and the full segment represents the conventional use (21 EE/DNG hormone-ingesting days+7 hormone-free days).

FIG. 5 shows the concentration of the anticoagulatory parameter antithrombin activity as a function of the time of use of the oral contraceptives, the control cycle indicating the concentration of antithrombin activity when no oral contraceptive is used. In the figure, the striped segment represents the uninterrupted use during the long cycle (84 EE/DNG hormone-ingesting days+7 hormone-free days) and the full segment represents the conventional use (21 EE/DNG hormone-ingesting days+7 hormone-free days).

FIG. 6 shows the concentration of the anticoagulatory parameter free protein S as a function of the time of use of the oral contraceptives, the control cycle indicating the concentration of protein S when no oral contraceptive is used. In the figure, the striped segment represents the uninterrupted use during the long cycle (84 EE/DNG hormone-containing days+7 hormone-free days) and the full segment represents the conventional use (21 EE/DNG hormone-ingesting days+7 hormone-free days).

FIG. 7 shows the concentration of the anticoagulatory parameter protein C activity as a function of the time of use of the oral contraceptives, the control cycle indicating the concentration of protein C activity when no oral contraceptive is used. In the figure, the striped segment represents the uninterrupted use during the long cycle (84 EE/DNG hormone-ingesting days+7 hormone-free days) and the full segment represents the conventional use (21 EE/DNG hormone-ingesting days+7 hormone-free days).

FIG. 8 shows the concentration of the profibrinological parameter plasminogen as a function of the time of use of the oral contraceptives, the control cycle indicating the concentration of plasminogen when no oral contraceptive is used. In the figure, the striped segment represents the uninterrupted use during the long cycle (84 EE/DNG hormone-ingesting days+7 hormone-free days) and the full segment represents the conventional use (21 EE/DNG hormone-ingesting days+7 hormone-free days).

FIG. 9 shows the concentration of the profibrinological parameter tissue plasminogen activator (t-PA) activity as a function of the time of use of the oral contraceptives, the control cycle indicating the concentration of t-PA activity when no oral contraceptive is used. In the figure, the striped segment represents the uninterrupted use during the long cycle and the full segment represents the conventional use of ethinylestradiol/dienogest

FIG. 10 shows the concentration of the antifibrinological parameter plasminogen activator inhibitor-1 antigen (PAI-1-antigen) as a function of the time of use of the oral contraceptives, the control cycle indicating the concentration of PAI-1 when no oral contraceptive is used. In the figure, the striped segment represents the uninterrupted use during the long cycle (84 EE/DNG hormone-ingesting days+7 hormone-free days) and the full segment represents the conventional use (21 EE/DNG hormone-ingesting days+7 hormone-free days).

FIG. 11 shows the concentration of the plasmin-antiplasmin complex (PAP), the marker for thrombin and fibrin formation, as a function of the time of use of the oral contraceptives, the control cycle indicating the concentration of PAP when no oral contraceptive is used. In the figure, the striped segment represents the uninterrupted use during the long cycle (84 EE/DNG hormone-ingesting days+7 hormone-free days) and the full segment represents the conventional use (21 EE/DNG hormone-containing days+7 hormone-free days).

FIG. 12 shows the concentration of the thrombin-antithrombin complex (TAT), the marker for thrombin and fibrin formation, as a function of the time of use of the oral contraceptives, the control cycle indicating the concentration of TAT when no oral contraceptive is used. In the figure, the striped segment represents the uninterrupted use during the long cycle (84 EE/DNG hormone-ingesting days+7 hormone-free days) and the full segment represents the conventional use (21 EE/DNG hormone-ingesting days+7 hormone-free days).

In a prospective, randomized comparative study, the effects of the conventional treatment with a single-phase combination of 30 μg of ethinylestradiol and 2 mg of DNG (EE/DNG) (21+7 days) on various procoagulatory, anticoagulatory, profibrinolytic and antifibrinolytic factors, markers for thrombin and fibrinium set and clotting tests, were compared with the effects of a long-cycle use [extended intake time of the hormone-containing daily dose units, uninterrupted use (84+7 days)] of EE/DNG. The points in time selected for the evaluation were 3 and 12 months.

### Material and Methods

1. Study Design

After a preliminary investigation of 88 healthy women between the ages of 20 and 33 years who wanted protection from pregnancy, 80 subjects with regular menstrual cycle and without contraindications to the use of oral contraceptives were included into the prospective, randomized study. These women had not taken any hormonal medications for at least four weeks preceding the study and were taking no drugs known to have an influence on the effects of oral contraceptives.

During (after 3 months) and at the end of the study (after 12 months), the test subjects were subjected to a general and gynecological examination including cervical cytology and a pregnancy test as well as to a determination of general laboratory parameters for safety.

After the control cycle, the test subjects were randomized and received ethinylestradiol/dienogest either conventionally (13 cycles of 21 treatment days and 7 days without hormones) or in a long cycle (4 long cycles of 84 days of uninterrupted intake and 7 days without hormones).

Blood samples were taken on Day 21-26 of the control cycle and on Day 82-84 of the first and fourth long cycle (in 3^{rd} and 12^{th} month) or on Day 19-21 of the third and thirteenth conventional cycle (in 3^{rd} and 12^{th} month). The blood samples were taken in the morning (between 8:00 and 10:00 a.m.) after natural overnight fasting.

Each test subject maintained a cycle log in which the tablet intake and any bleeding events were recorded daily. Within 28 days after the end of the treatment, a final examination was performed (a general and a gynecological examination including cervical cytology, laboratory tests for safety and pregnancy test). On the examination days, the test subjects were asked about any undesirable events.

2. Laboratory Methods

For the analysis of the hemostasis parameters, the blood samples were centrifuged and the citrated plasma was stored at −70° C. up to the time of the examination.

Procoagulatory Parameters

Fibrinogen was measured coagulometrically by the Claus method using a test kit, the factor VII activity (VIIc) was determined by a (single-stage) clotting test using plasma with a factor VII deficiency, activated factor VII (VIIa) was measured by a clotting test (Staclot® VIIa-rTF), factor VII antigen was determined by use of a commercially available heterogeneous enzyme immunoassay (ELISA, Asserachrom® VII:Ag) and factor VIII activity (VIIIc) was determined by a (single-stage) clotting test using plasma with a factor VIII deficiency.

### Anticoagulatory Parameters

Antithrombin antigen was determined nephelometrically with N antiserum against antithrombin III, the antithrombin activity was determined by means of a chromogenic substrate (Coamatic® LR Antithrombin, protein C antigen by an ELISA (REAADS® protein C antigen test kit), protein C activity by means of a chromogenic substrate (Coamatic® protein C) and free protein S and total protein S by an ELISA (REAADS® protein S antigen test kit).

Profibrinolytic Parameters

Plasminogen was determined by means of a chromogenic substrate (Coamatic® Plasminogen), the tissue plasminogen activator (t-PA) antigen by an ELISA (t-PA antigen ELISA) and the t-PA activity by an ELISA (t-PA Actibind®).

Antifibrinolytic Parameters

The plasminogen activator inhibitor-1-antigen (PAI-1-antigen) was determined by an ELISA (PAI-1 Actibind®).

Markers for Thrombin and Fibrin Formation

The thrombin-antithrombin complex (TAT) was measured by means of an ELISA (Enzygnost TAT micro), the plasmin-α2 antiplasmin complex (PAP) by an ELISA (PAP micro), the prothrombin fragments 1+2 by an ELISA (Enzygnost F 1+2 micro) and the D dimers by an ELISA (Dimer test gold).

Clotting Tests

The prothrombin time (PT) (Quick test) and the activated partial thromboplastin time (APTT) were measured by clotting tests (Thromborel® S and Pathromtin SL).

3. Statistical Analysis

The size of the population was set at 60 test subjects, namely 30 per study branch. This population size was used frequently to describe the hemostatic changes occurring during the intake of hormonal contraceptives. All variables were examined based on FAS (full analytical set). The study parameters were evaluated descriptively by comparing the averages on each evaluation date and their changes with time. Missing data were not replaced. The changes occurring with time (namely the absolute changes of the intra-group comparisons between the evaluation dates) were evaluated by the sign rank test. The comparisons between the groups were evaluated by Wilcoxon's rank sum test. The significance level was p=0.05.

Results

1. Test Subject Distribution

Sixty test subjects were randomized for the treatment with ethinylestradiol/dienogest either over 13 conventional cycles (21+7 days) or over 4 long cycles (84+7 days). One test subject randomized for the conventional regimen became pregnant during the control cycle and interrupted the study before taking the first tablet so that this group was reduced to n=29. Two women interrupted the study prematurely during the first long cycle, one because of irregular bleeding and the other because of tinnitus. Hence, 57 test subjects continued participating in the study until the end (conventional intake n=29, long cycle n=28). The statistical analysis was examined on the basis of FAS (full analytical set) (n=59). The two treatment groups were comparable in terms of the baseline data of age and average body mass index. The average BMI did not change substantially during the treatment. Also, no relevant change in average blood pressure or pulse rate was observed during the study. 2. Procoagulatory Factors

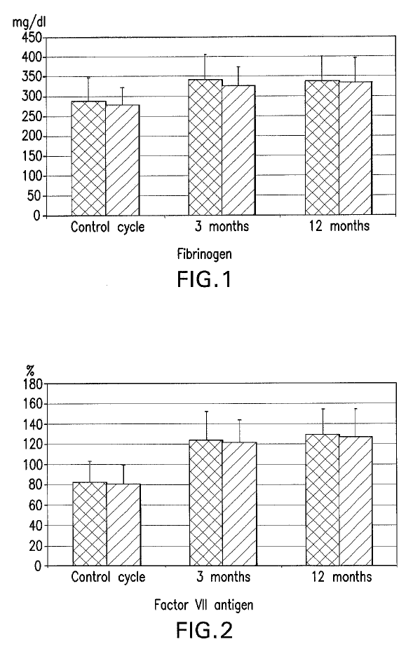

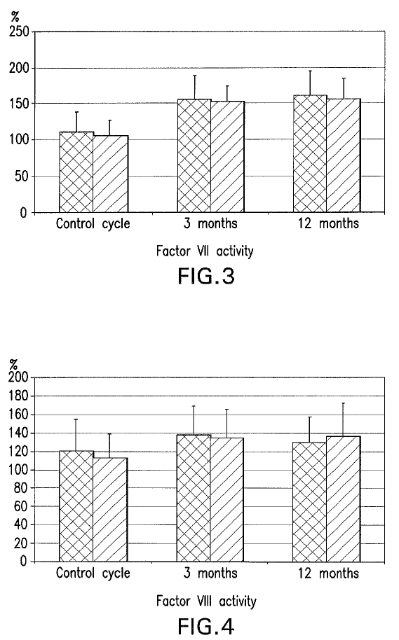

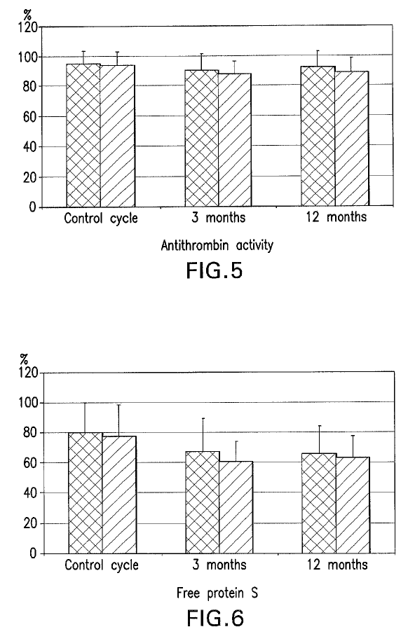

At no time was a significant difference in concentration or activity of procoagulatory factors found between the two treatment groups. In both groups, fibrinogen was significantly higher, namely by about 20% in the 3rd and 12th month (FIG. 1). Both during the conventional intake and during the long-cycle treatment, the factor VII antigen (FIG. 2), FVII activity (FIG. 3) and activated FVII increased by 30-60%. The increase observed at the 3rd month showed only a slight further increase up to the 12th month of treatment. As regards the factor VIII activity (FIG. 4), a 15-20% increase was noted at the 3rd month in both groups. After that, the activity during conventional intake tended to drop, but did not change in the long-cycle group. Conventional intake of ethinylestradiol/dienogest produced a slight increase in concentration of prothrombin fragment 1+2 while a moderate increase took place in the long-cycle regimen, but the difference between the two treatment groups was not significant. The plasma level of TAT (FIG. 12) showed in both groups large inter-individual differences in the control cycle and during treatment with ethinylestradiol/dienogest. Compared to the control cycle, the changes, however, were not significant.

3. Anticoagulatory Factors

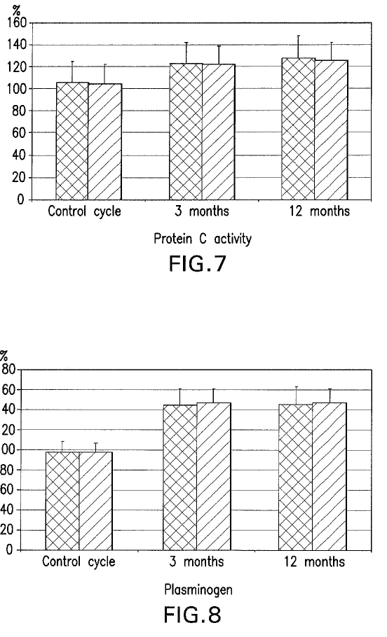

In both groups, a marginal drop in concentration and activity of antithrombin was noted at 3 and 12 months of treatment. Free protein S (FIG. 6) and total protein S were reduced by about 20%. By contrast, protein C antigen and protein C activity increased in both treatment groups by 15-20% after 3 and 12 months of treatment (FIG. 7).

4. Fibrinolysis Variables

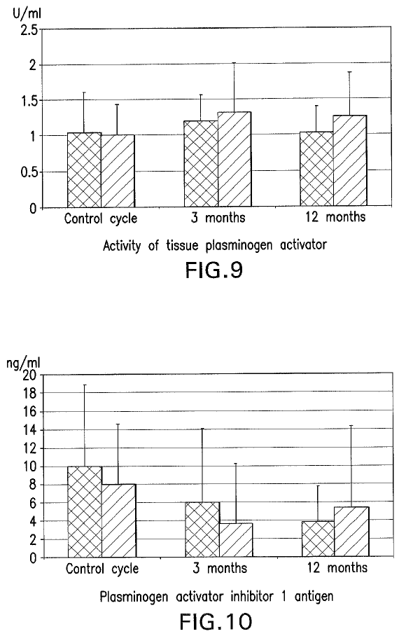

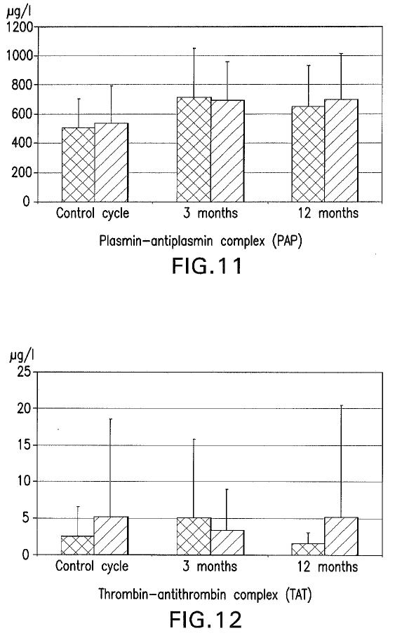

After 3 months of treatment, a comparable increase in the plasminogen level (about 50%) (FIG. 8) was noted in both groups. This level did not change after the 12th month. The t-PA antigen dropped by 30% during both the conventional and the long-cycle intake. In the conventional group, the t-PA activity (FIG. 9) was 15% higher only after 3 months, whereas in the long-cycle group, after 3 and 12 months, it was 25% higher. A marked increase was noted in the two fibrinolysis activation markers, 30-40% in the case of PAP (FIG. 11) and 20-55% in the case of the D dimers, but because of the large inter-individual variations the two treatment groups showed no significant differences. In both groups, the PAP-1 level (FIG. 10) dropped by 40-50% after 3 months and by 35-80% after 12 months.

5. Overall Clotting Tests

Both clotting tests were affected only slightly; while the prothrombin time increased by about 10%, the APTT showed an acceleration of 10%.

In this randomized study, the effects of conventional intake and of the long-cycle use of an oral contraceptive on different hemostasis parameters have for the first time been compared directly to one another.

Surprisingly, it was found that after 12 months there were no significant differences between the conventional (21+7 days) and the long-cycle treatment regimen (84+7 days). Both groups showed a significant increase in fibrinogen, factor VII and factor VIII, protein C, plasminogen and t-PA activity and a decrease in antithrombin, protein S, t-PA antigen and PAT-1 antigen. In parallel with this, the average values over time of prothrombin fragment 1+2, D-dimers and PAP increased.

At the end of the use, both intake regimens resulted in a comparable, significant increase in fibrinogen (20%), factor VII antigen (50-60%), factor VII activity (45%), activated factor VII (30-45%) and factor VIII activity (10-20%), and also a smaller but significant decrease in concentration and activity of antithrombin, a 20-25% decrease in total and free protein S and a 15-20% increase in concentration and activity of protein C. The thrombin-antithrombin complex showed no significant changes. A significant increase in pro-thrombin fragment 1+2 of about 25% took place only in the long-cycle group, but this effect was not significantly different from the one observed during conventional treatment. At both points in time, plasminogen was found to have increased by 50% in both groups, and the activity of the tissue plasminogen activator (t-PA) was found to have increased by 15% in the conventional group and by 25-30% in the long-cycle group. A decrease in t-PA antigen of about 30% and of the plasminogen activator inhibitor-1 antigen (PAI-1 antigen) of 40-60% was noted in both groups. The concentrations of the plasmin-antiplasmin complex increased by 30- 40% and those of the D dimers by 20-55%. The prothrombin time increased by about 10% while the activated partial thromboplastin time dropped by about 10%.

It is shown that the estrogen-induced changes in hemostasis reach a steady state within a few weeks and that the effects noted after 3 and 12 months of treatment do not differ substantially between long-cycle intake and conventional intake of ethinylestradiol/dienogest.

1. Process for producing a single-phase pharmaceutical preparation for limiting/reducing the risk of deep venous thrombosis in combination with oral contraception, characterized in that a contraceptive combination of 2.0 mg of 17-α-cyanomethyl-17-β-hydroxyestra-4,9-dien-3-one (dienogest) and 0.030 mg of 17-α-ethinylestradiol (ethinylestradiol) or 2.0 mg of dienogest and 0.020 mg of ethinylestradiol is used in n×21 daily dose units and the n×21 units being followed by at the most 7 daily hormone-free or placebo-containing units, with n equal to 2, 3, 4, 5, 6, 7, 8, 9, 10, 11, 12, 13, 14, 15, 16 and 17.

2. Process as defined in claim 1, characterized in that the drug form of the single-phase pharmaceutical preparation is a film tablet consisting of a tablet core containing part of the total dienogest to be released in retarded manner and a film coating containing part of the total dienogest to be released in non-retarded (fast) manner and the total amount of ethinylestradiol to be released in non-retarded (fast) manner.

3. Process as defined in claim 1, characterized in that at least 10%, and preferably 30%, of the dienogest is dissolved out of the tablet core in retarded manner after more than 30 minutes as determined by the dissolution test using 37° C. water as the dissolution medium and a rotation rate of 50 rpm.

4. Kit characterized in that it contains n×21 daily dose units with n equal to 2, 3, 4, 5, 6, 7, 8, 9, 10, 11, 12, 13, 14, 15, 16 and 17 and n×21 of a maximum of 7 daily hormone-free or placebo-containing dose units of a single-phase pharmaceutical preparation with a combination of 2.0 mg of dienogest and 0.030 mg of ethinylestradiol or 2.0 mg of dienogest and 0.020 mg of ethinylestradiol together with one or more pharmaceutically acceptable auxiliary agents/carriers and wherein the single-phase pharmaceutical preparation for limiting/reducing the risk of deep venous thrombosis is used in combination with oral contraception.

5. Kit as defined in claim 4, characterized in that the number of daily hormone-free or placebo-containing dose units is 3, 4, 5, 6 and 7.

6. Kit as defined in claim 4, characterized in that the number of daily dose units of the single-phase pharmaceutical preparation with the combination of dienogest and ethinylestradiol amounts to 84 and that of the hormone-free or placebo-containing daily dose units amount to 7 so that the total number of cycle days per year is 4 (n×21 plus 7) where n equals 4.

7. Kit as defined in claim 4, characterized in that the drug form of the single-phase pharmaceutical preparation is a film tablet consisting of a tablet core containing part of the total amount of dienogest to be released in retarded manner and a film coating containing part of the total dienogest to be released in non-retarded (fast) manner and the total amount of ethinylestradiol to be released in non-retarded (fast) manner.

8. Kit as defined in claim 4, characterized in that from the drug form at least 10%, and preferably 30%, of dienogest is dissolved out of the tablet core in retarded manner after more than 30 minutes as determined by the dissolution test using 37° C. water as the dissolution medium at a rotation rate of 50 rpm.
